(19) United States
(12) Patent Application Publication
(10) Pub. No.: US 2005/0065493 A1
(43) Pub. Date: Mar. 24, 2005

(54) Feminine hygiene article

(76) Inventor: Carolina H. Henderson, Lafayette, CO (US)

Correspondence Address: MARIAN J. FURST, 988 SYCAMORE AVENUE, BOULDER, CO, 80303, US

(21) Appl. No.: 10/671,174

(22) Filed: Sep. 24, 2003

(51) Int. Cl. A61F 13/15; A61F 13/20

(52) U.S. Cl. 604/385.01

(57) ABSTRACT

A feminine hygiene article comprising a narrow, elongated absorbent article that is placed between the legs of the user with one end positioned between the user's buttocks and the other end extending forward under the user's urogenital area. Preferably, the article is easily disposable.

### FIELD OF THE INVENTION

The present invention relates generally to a feminine hygiene article, and more particularly to a feminine hygiene article for absorption of bodily fluids with improved comfort and convenience for use while the user is sleeping.

### BACKGROUND OF THE INVENTION

Many feminine hygiene products have been developed for absorption of bodily fluids, such as menstrual fluids, other vaginal discharges, and urine, such as in cases of stress incontinence. Despite the abundance of such products, women often find them unsatisfactory for use under certain conditions, including during the night while the user wants to relax and sleep in comfort.

Tampons, which are inserted into the user's vagina, are generally comfortable, but it is well documented that their use can result in serious infections, such as toxic shock syndrome, if they are left in place for time periods as long as an entire night asleep. Further, tampons are also known to leak menstrual fluids onto night clothes and bedding. In addition, some women find their use awkward or distasteful. Also, there are some medical circumstances under which it is undesirable to insert objects, such as tampons, into the vagina. Examples of such conditions include infections and minor surgical procedures that cause vaginal discharge.

Sanitary napkins and pads are bulky and uncomfortable when used. Previously, sanitary napkins were designed with tails for attachment to a belt that encircled the user's abdomen. This arrangement held the napkins in close proximity to the user's urogenital area, but the belt and bulky napkins were uncomfortable, and the napkins tended to bunch up between the wearer's legs and then leak around the sides onto panties and other clothing and bedding. The napkins and pads in current use generally are attached to the user's panties and may not be in close proximity to the vaginal area while the user is in a horizontal position, such as for sleeping. Under these circumstances, fluids may not be absorbed properly by the napkin or pad and may leak onto clothing and bedding. In recent years, a number of pads or napkins, designed for overnight use, have been introduced commercially in attempts to solve this problem. However, these pads are thicker and longer than pads intended for daytime use, and they sometimes have stiff, fluid-impervious backings. Commercially available overnight pads have dimensions as long as about 12 inches, widths of about 3.5 inches, and thicknesses of one-half to three-fourths inch. These features often cause discomfort while these pads are used. Further, the fluid-impervious backings may rub the user's skin uncomfortably when the pads are compressed laterally between the user's legs during use.

Panty liners are less bulky and more comfortable than thicker pads, but they may not be sufficiently absorbent for heavier menstrual flow, and they also have the disadvantage that they may allow fluid to leak onto the user's clothing and onto bedding.

Other devices, such as interlabial devices, have also been developed, but these devices tend to be difficult to position properly for effective use and uncomfortable when used.

Thus, there is a need for a comfortable feminine hygiene article that is suitable for absorption of menstrual and other bodily fluids, particularly during the night when the user desires comfort and close contact with the urogenital area but does not want to risk the chance of infection with the use of tampons. In addition, there is a need for an article that is easily positioned for use and easily removed for disposal after use.

### SUMMARY OF THE INVENTION

One embodiment of the present invention comprises a feminine hygiene article for absorption of bodily fluids. The article comprises an elongated absorbent article having a front end, a back end, a length between the front and back ends sufficient to extend from a point between the user's buttocks to a point beyond the user's urogenital area, a height less than about 2 inches, and a width sufficiently small to fit between the user's buttocks and legs without causing significant discomfort. The article is formed such that said back end can be inserted between and retained in position by a user's buttocks while said front end extends forward beyond the user's urogential area, and the article does not include an intravaginal portion. Preferably, the feminine hygiene article comprises a soft material that is substantially non-irritating to human skin and membranes in the genital area. The material may be selected from bonded fabrics, woven fabrics, cellulose, paper, and combinations thereof. There may be a plurality of layers of the material. Preferably, the material is biodegradable and/or can be flushed into a sewer system. The material may be folded or rolled into an elongated coil, and the coil may be twisted or creased at a position between the front and back ends. Preferably the article has a length between about 6 inches and about 10 inches, a width less than about 1.5 inches, and/or a height between about 0.5 inches and about 1.5 inches. The article may be flexible and conformable to the shape of the body area where it is used.

Another embodiment of the present invention comprises a feminine hygiene kit. The kit includes an elongated absorbent article having a front end, a back end, a length between the front and back ends sufficient to extend from a point between the user's buttocks to a point beyond the user's urogenital area, a height less than about 2 inches, and a width sufficiently small to fit between the user's buttocks and legs without causing significant discomfort, wherein said article is formed such that said back end can be inserted between and retained in position by a user's buttocks while said front end extends forward beyond the user's urogential area. The absorbent article does not include an intravaginal portion. In addition, the kit includes an absorbent pad held in placed between the user's panties and her urogenital area. The absorbent pad may be selected from panty liners, mini-pads, maxi-pads, and sanitary napkins.

### BRIEF DESCRIPTION OF THE DRAWINGS

These and other features, aspects, and advantages of the present invention will become better understood with reference to the following description, appended claims, and accompanying drawings, where:

FIG. 1a is a perspective view of three stacked sheets of an absorbent material for use in the present invention, and FIGS. 1b, c, and d are perspective views showing how the stack is folded in accordance with one embodiment of the present invention;

FIG. 2 is a rear view of a portion of a woman's body with the embodiment of FIG. 1 in place;

FIG. 3 is a front view of a portion of a woman's body with the embodiment of FIG. 1 in place;

FIG. 4 is another view of a portion of woman's body with the embodiment of FIG. 1 in place;

FIG. 5 is a perspective view of another embodiment of the present invention, with FIG. 5a showing several rolled sheets of a starting material, and FIG. 5b showing how the rolled sheets are formed into an article in accordance with the present invention;

FIG. 6 is a cutaway view of a portion of a female body with a side view of the embodiment of FIG. 5; and

FIG. 7 shows a portion of a woman's body with the embodiment of FIG. 5 in place.

### DESCRIPTION OF THE PREFERRED EMBODIMENTS

The present invention comprises an article suitable for absorption of menstrual and other bodily fluids discharged from the urogenital area. The article is particularly suitable for use while the user is asleep or otherwise relatively inactive and in a more or less horizontal position. The present invention provides improved comfort and reliability in comparison to previously available articles, such as tampons, sanitary napkins, and interlabial devices. While being highly absorbent, it is also soft, flexible, and non-bulky. Further, it does not require the inclusion of a relatively stiff, waterproof barrier. Although the following discussion refers to the use of the present invention for absorbing menstrual flow, it is also useful for absorbing other fluids discharged from the urogenital area. These fluid may include other vaginal discharges, fluids resulting from sexual intercourse, or relatively small amounts of urine, such as might be released in cases of stress incontinence.

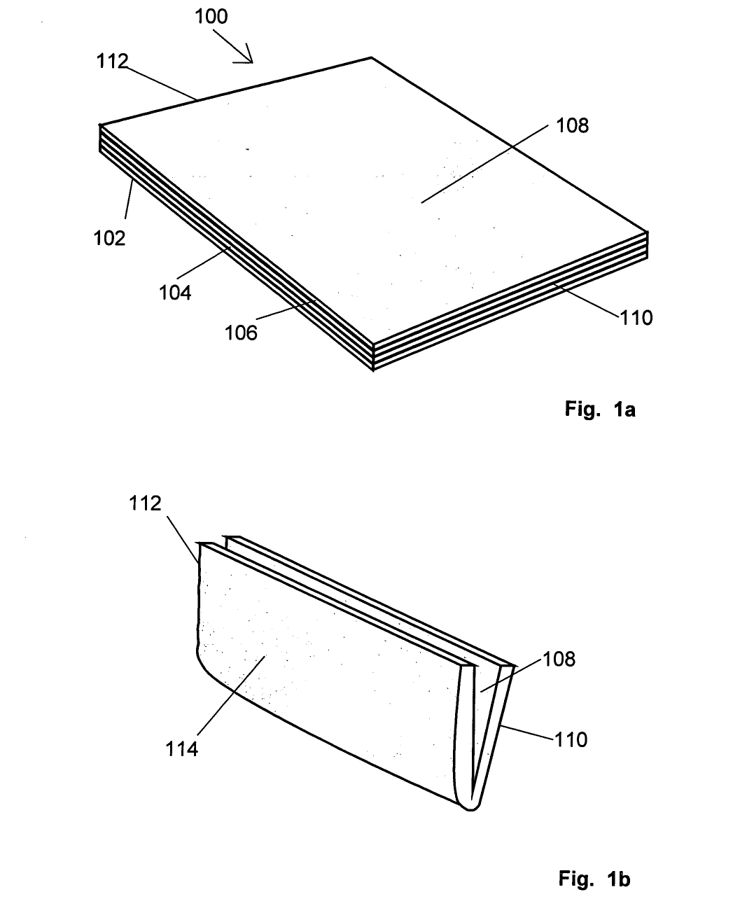

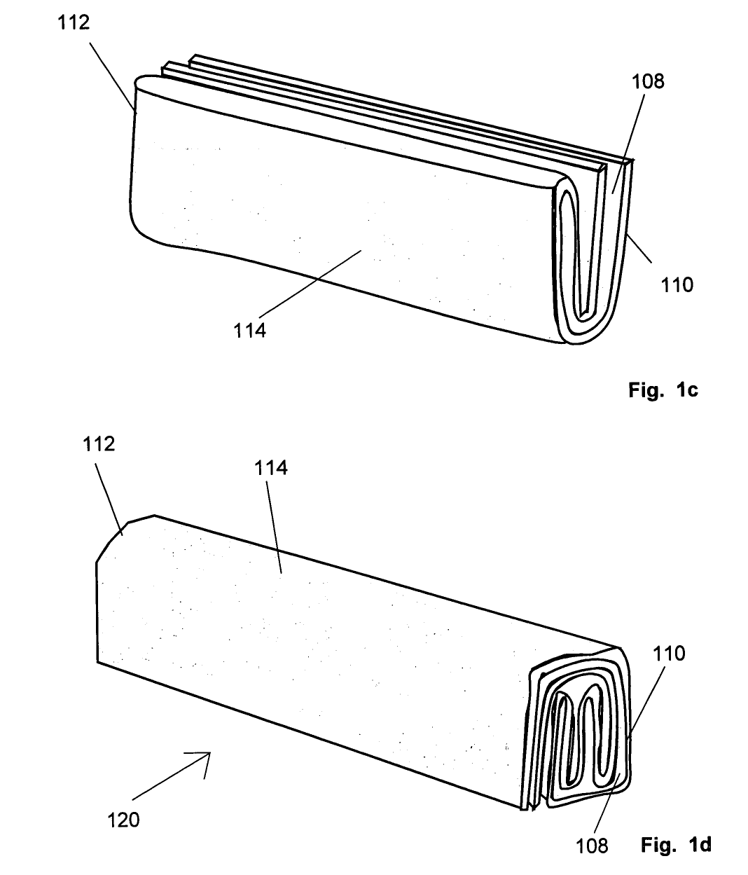

The article in accordance with the present invention comprises multiple layers of a soft, absorbent material formed into a narrow elongated shape. Multiple layers or sheets 102, 104, and 106 of an absorbent material 108 can be used, as illustrated in FIG. 1a. Although three layers are shown in FIG. 1, any number could be used, depending on the thickness of the individual layers and the overall absorbency desired. Preferably, the material is selected so that it will not irritate sensitive skin and membranes with which it comes into contact. It may comprise woven or bonded fabric, paper, cellulose, or combinations thereof. Preferably, the material is disposable. More preferably, the material comprises paper that can be flushed into a sewage system without a major likelihood of clogging the system.

One embodiment of the present invention can be formed as shown in FIGS. 1a-d. Although it is contemplated that the article may have multiple layers as described above and shown in FIG. 1a, only 1 layer is shown in FIGS. 1b-d for improved clarity of the drawings. A rectangular sheet or stack of sheets 100 of an absorbent material is initially flat, as shown in FIG. 1a, with top surface 108 and ends 110 and 112. Stack 100 is folded approximately in half lengthwise as shown in FIG. 1b, and then successively folded approximately in half twice again lengthwise as shown in FIGS. 1c and 1d to form article 120. The top and bottom surfaces of stack 100 are indicated as surfaces 108 and 114, respectively, after folding.

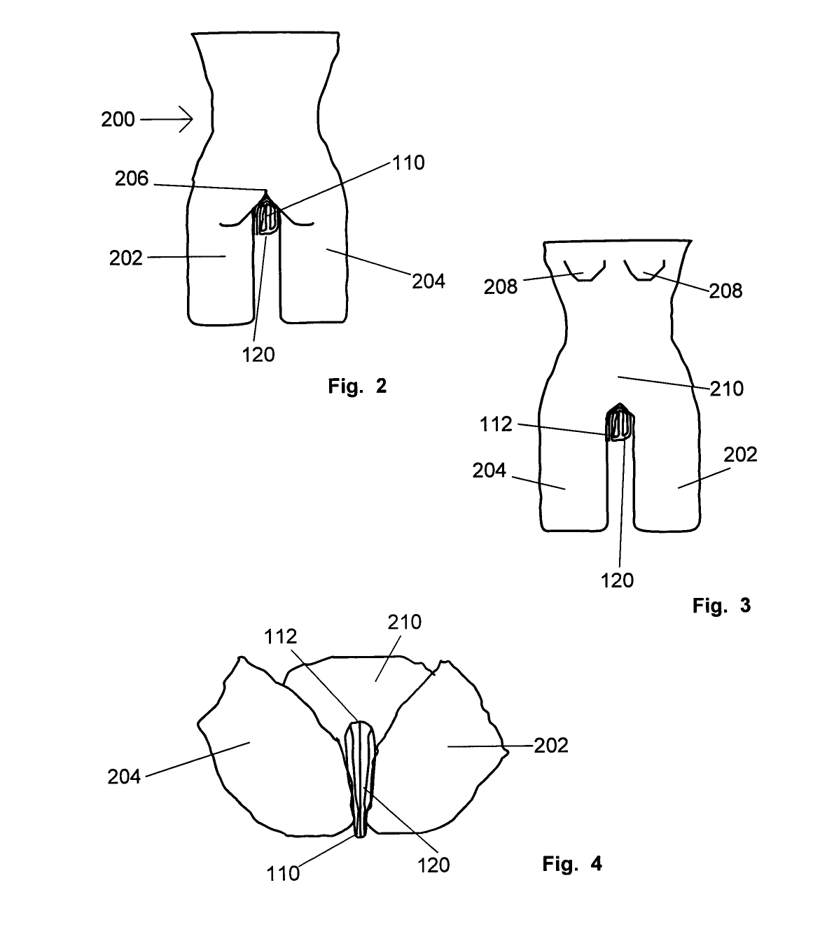

In use, the article 120 is inserted between the user's legs, with end 110 between the user's buttocks and end 112 extending forward beyond the vaginal and urethral openings. FIG. 2 shows a portion of a woman's body 200 from the rear, with left leg 202, right leg 204, and a space between her buttocks 206. End 110 of article 120 is positioned between the buttocks. The front of the woman's body 200 is shown in FIG. 3, with breasts 208 visible. End 112 of article 120 can be seen between the woman's legs 204 and 202. The woman's abdomen is indicated as 210. FIG. 4 shows another view of article 120, looking upwards from below the woman's body. It should be noted that the end 110 is held in place by friction and pressure from the buttocks. End 112 need not be inserted between the labia of the user, and, as shown in FIGS. 3 and 4, it is not.

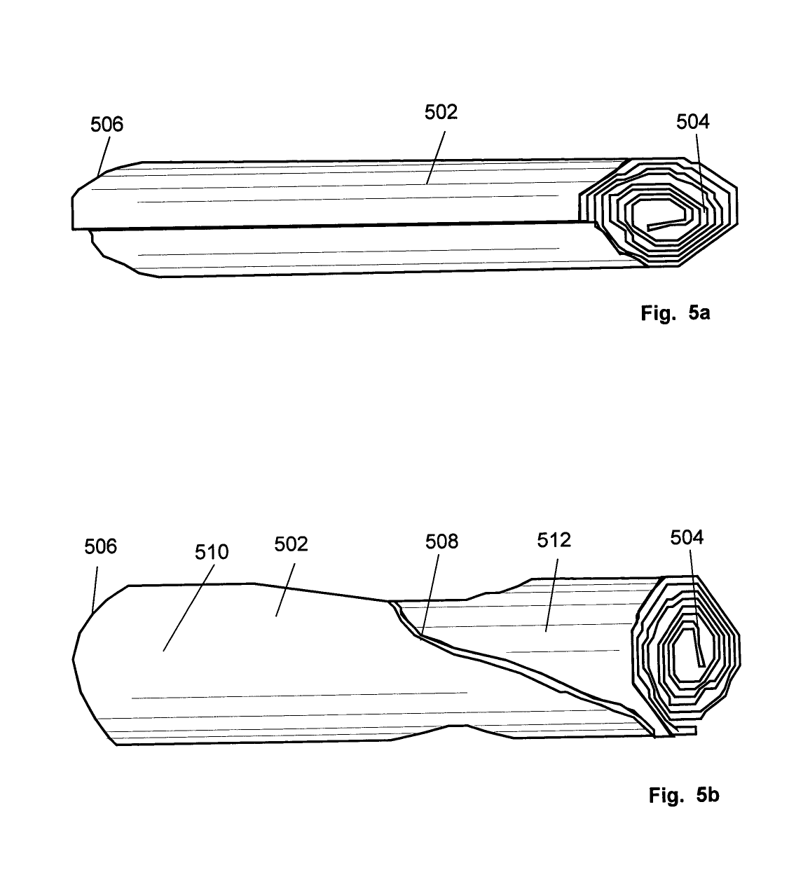

Another embodiment of the present invention is shown in FIG. 5. A sheet or stack of sheets of material is coiled loosely into an approximately cylindrical shape 502, as shown in FIG. 5a. The cylindrical coil 502 may then be twisted at a position between end 504 and 506 to prevent it from uncoiling. Preferably, the twisted section 508 is closer to end 504 than to end 506, and most preferably twisted section 508 divides the length of article 500 into two portions, with a first portion 510 approximately twice as long as a second portion 512.

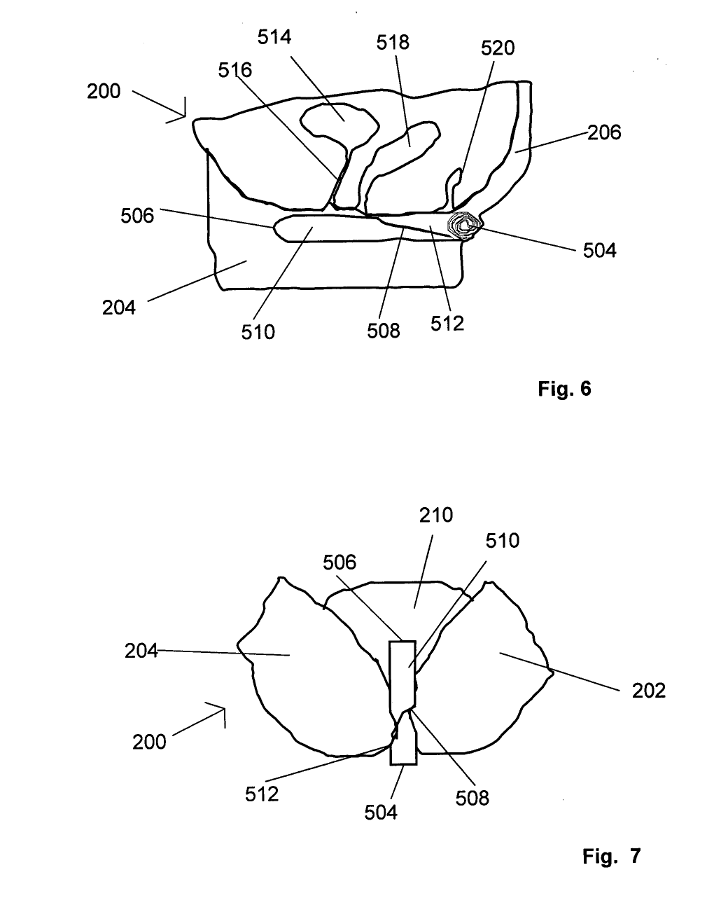

In use, coil 502 is positioned between the user's legs, with shorter section 512 between the user's buttocks and longer section 510 extending between the user's legs past the vaginal and urethral openings, as shown in FIGS. 6 and 7. Referring to FIG. 6, a cutaway view of a portion of the woman's body 200 is shown, with a portion of right leg 204 extending downward. Several internal organs are shown, including bladder 514, urethra 516, vagina 518, and anus 520. The shorter portion 512 of article 510 is inserted between the buttocks, and the longer portion 510 extends forward. Coil 502 can be seen to extend from near the opening of the anus 520 to a point forward of the opening of urethra 516, and longer portion 510 is not inserted between the user's genital labia or into the user's vagina.

In view of how the article is used, the sheets of the starting material preferably have a length selected so that the article can be retained in position between the buttocks and can extend forward beyond the urogenital area. A length between about 7 inches and about 10 inches is compatible with the anatomy of most women. The height and thickness of the article after folding or rolling are each preferably between about 0.5 and about 1.5 inches. As illustrated in FIGS. 1 and 5, the layers are loosely folded or coiled to maximize the comfort and fluid absorption capacity of the feminine hygiene article. If the material is sufficiently absorbent, the layers could be folded or coiled more tightly.

The article of the present invention is particularly suitable for use while resting or sleeping in a reclining position. The article stays in position with horizontal orientation of user and minimal motion. The increased comfort relative to other feminine hygiene articles allows the user to relax and fall asleep or rest comfortably. Further, it eliminates messy leakage from thicker pads that are attached to or held in place by the user's panties. It also eliminates the risk of infection from overnight use of tampons. The article in accordance with the present invention may be disposable. Preferably, the material from which it is made comprises paper that is flushable in ordinary sewer and septic systems. Thus, when the user is finished with it, it can be easily disposed of in an ordinary commode, eliminating the additional handling required for disposal of most other feminine hygiene articles. The article is also lightweight and compact, allowing a user with an active lifestyle to carry it easily.

Nevertheless, the present invention may also include a thin panty liner or thicker pad, if desired, to provide additional absorption and protection from leakage of the article between the user's legs. Further, the use of a panty liner or thicker pad provides protection while the user disposes of the article and prepares for her daily morning routine. Anecdotal records indicate that although the extra fluid absorption provided by a panty liner was actually needed in a very small percentage the times the article was used, the liner provided psychological reassurance that the woman was protected during the night. It has been observed that a thin panty liner is adequate for most cases, maintaining the benefit of improved comfort relative to thicker pads.

The following nonlimiting examples are provided to further illustrate the invention.

### EXAMPLE 1

A paper towel was folded successively to obtain an elongated article with a width of about 1 inch. The folded towel was then inserted between a menstruating woman's legs, with one end between her buttocks and the rest of the towel extending forward underneath and close to the woman's pubic area. The woman then slept overnight with the folded towel in that position. She awoke in the morning to find that the towel had absorbed her menstrual flow during the night without any fluid leaking to stain her panties, pajamas, or bedding. However, the towel irritated the skin between her buttocks and the membranes in her genital area.

### EXAMPLE 2

A commercially available panty liner was folded in half lengthwise with the absorbent side facing outward and the plastic-lined water-proof side to the interior of the fold. The folded liner was then inserted between the legs of a menstruating woman, with one end between her buttocks and the other end adjacent her urogenital area. The woman then retired to bed for the night. The plastic liner was stiff and uncomfortable, and the folded panty liner did not absorb all of her menstrual fluid during the night.

### EXAMPLE 3

Another brand of a commercially available panty liner was folded an inserted between the legs of a menstruating woman as described for Example 2. This liner had a softer water-impermeable plastic surface than the type used in Example 2. The liner absorbed more fluid than the one used in Example 2, but it became stiff and uncomfortable as it absorbed fluid during the night.

### EXAMPLE 4

A terry cloth washcloth was folded lengthwise repeatedly and inserted between the legs of a menstruating woman as described for Example 1. The washcoth absorbed the menstrual fluids overnight, but it was too bulky for comfort. The washcloth was difficult to launder for re-use, and the tester considered it to be too expensive for disposal after a single use.

### EXAMPLE 5

Three Kleenex® Ultra Soft-Doux tissues (manufactured by Kimberly-Clark Corporation of Neenah, Wis. 54596, USA), each measuring 8.6 inches by 8.4 inches and comprising 3 plys of soft absorbent paper, were stacked on top of one another and folded successively into halves, quarters, and eighths, as illustrated in FIG. 1, to form an elongated article 8.4 inches long and about 1 inch high. The folded tissues were inserted between the legs of a menstruating woman, with one end between her buttocks and the other end under and just in front of her pubic area. The woman then slept comfortably overnight with the folded tissues in that position. She awoke in the morning to find that the article had absorbed her menstrual flow during the night without any fluid leakage to stain her panties, pajamas, or bedding, and without irritating her skin. The article was flushed down the commode for disposal. This process was repeated numerous times, without leakage of menstrual fluid onto any clothing or bedding and without obstruction of sewer lines.

### EXAMPLE 6

Four Kleenex® Ultra Soft-Doux tissues (manufactured by Kimberly-Clark Corporation of Neenah, Wis. 54596, USA), each measuring 8.6 inches by 8.4 inches and comprising 3 plys of soft absorbent paper, were rolled loosely into an 8.4-inch long coil, as illustrated in FIG. 5a. The roll of tissues was twisted about one-third of the way from one end to the other to maintain its coiled structure, as shown in FIG. 5b. The coil was then inserted between the legs of a menstruating woman, with the shorter portion between her buttocks and the longer portion extending forward to a position under and just in front of her pubic area, as illustrated in FIGS. 6 and 7. The woman then slept comfortably overnight with the coil of tissues in that position. Upon awakening in the morning, she found that the article had absorbed her menstrual flow during the night without any fluid leaking to stain her panties, pajamas, or bedding, and without irritating her skin. The loose coil of tissues was more comfortable in place than the folded tissues described in Example 3. The article was disposed of by flushing down the commode. This process was repeated numerous times, without menstrual fluid leaking onto any clothing or bedding, and without obstructing sewer lines.

The foregoing description is considered as illustrative only of the principles of the invention. Further, since numerous modifications and changes will readily occur to those skilled in the art, it is not desired to limit the invention to the exact construction and process shown and described above. Accordingly, all suitable modifications and equivalents may be resorted to falling within the scope of the invention.

1. A feminine hygiene article for absorption of bodily fluids, comprising:
an elongated absorbent article having a front end, a back end, a length between said front and back ends sufficient to extend from a point between the user's buttocks to a point beyond the user's urogenital area, a height less than about 2 inches, and a width sufficiently small to fit between the user's buttocks and legs without causing significant discomfort;
wherein said article is formed such that said back end can be inserted between and retained in position by a user's buttocks while said front end extends forward beyond the user's urogential area, and does not include an intravaginal portion.

2. The feminine hygiene article of claim 1, comprising a soft material that is substantially non-irritating to human skin and membranes in the genital area.

3. The article of claim 2, wherein said material is selected from bonded fabrics, woven fabrics, cellulose, paper, and combinations thereof.

4. The article of claim 2 wherein said article comprises a plurality of layers of said material.

5. The article of claim 1, wherein said article comprises at least one folded layer.

6. The article of claim 1, wherein said article comprises at least one layer and is rolled into an elongated coil.

7. The article of claim 6, wherein said coil is twisted or creased at a position between said front and back ends.

8. The article of claim 1, wherein said article has a length between about 6 inches and about 10 inches.

9. The article of claim 1, wherein said article has a width less than about 1.5 inches.

10. The article of claim 1, wherein said article has a height between about 0.5 inches and about 1.5 inches.

11. The article of claim 1, wherein said article is flexible and can conform to the shape of the body area where it is used.

12. The article of claim 1, comprising a material that can be flushed into a sewer system.

13. The article of claim 1, comprising a material that is biodegradable.

14. A feminine hygiene kit, comprising:
an elongated absorbent article having a front end, a back end, a length between said front and back ends sufficient to extend from a point between the user's buttocks to a point beyond the user's urogenital area, a height less than about 2 inches, and a width sufficiently small to fit between the user's buttocks and legs without causing significant discomfort, wherein said article is formed such that said back end can be inserted between and retained in position by a user's buttocks while said front end extends forward beyond the user's urogential area, and does not include an intravaginal portion; and
an absorbent pad held in placed between the user's panties and her urogenital area.

15. The kit of claim 12, wherein said absorbent article is selected from panty liners, mini-pads, maxi-pads, and sanitary napkins.
